(19) United States
(12) Patent Application Publication
(10) Pub. No.: US 2026/0240753 A1
(43) Pub. Date: Aug. 20, 2026

(54) COSMETIC AND METHOD FOR PRODUCING EXTRACT OF GREEN ALGA CHLAMYDOMONAS REINHARDTII

(71) Applicant: HONDA MOTOR CO., LTD., Tokyo (JP)

(72) Inventors: Nozomi SHIOBARA, Saitama (JP); Shohei KINOSHITA, Saitama (JP)

(21) Appl. No.: 19/412,872

(22) Filed: Dec. 9, 2025

(30) Foreign Application Priority Data
Feb. 20, 2025 (JP) 2025-025671

(51) Int. Cl. A61K 8/9722; A61K 8/64; A61Q 19/00

(52) U.S. Cl. CPC A61K 8/9722; A61K 8/64; A61Q 19/00; A61K 2800/10

(57) ABSTRACT

A cosmetic containing: an extract of Chlamydomonas reinhardtii, the extract of the Chlamydomonas reinhardtii containing one or more dipeptides selected from the group consisting of glycine-leucine, leucine-glycine, glycine-isoleucine, and isoleucine-glycine.

This application is based on and claims the benefit of priority from Japanese Patent Application No. 2025-025671, filed on 20 Feb. 2025, the content of which is incorporated herein by reference.

### BACKGROUND OF THE INVENTION

### Field of the Invention

The present invention relates to a cosmetic and a method for producing an extract of Chlamydomonas reinhardtii.

### Related Art

Placenta has been attracting attention because of its ability to enhance natural healing power. Known dipeptides derived from pig placenta include glycine-leucine, leucine-glycine, glycine-isoleucine, and isoleucine-glycine.

Patent Document 1 describes a cosmetic composition containing a pig placenta extract.

On the other hand, Patent Document 2 describes a Honda DREAMO strain (accession number: FERM BP-22306) as an example of Chlamydomonas reinhardtii.
Patent Document 1: Japanese Unexamined Patent Application, Publication No. 2018-203628
Patent Document 2: PCT International Publication No. WO2017/217116

### SUMMARY OF THE INVENTION

However, the use of pig placenta does not allow the production of organic cosmetics.

An object of the present invention is to provide an organic cosmetic containing at least one dipeptide derived from pig placenta.
(1) A cosmetic containing: an extract of Chlamydomonas reinhardtii, the extract of the Chlamydomonas reinhardtii containing one or more dipeptides selected from the group consisting of glycine-leucine, leucine-glycine, glycine-isoleucine, and isoleucine-glycine.
(2) The cosmetic as descried in (1), wherein the Chlamydomonas reinhardtii is a Honda DREAMO strain (accession number: FERM BP-22306).
(3) A method for producing an extract of Chlamydomonas reinhardtii, the extract of the Chlamydomonas reinhardtii containing one or more dipeptides selected from the group consisting of glycine-leucine, leucine-glycine, glycine-isoleucine, and isoleucine-glycine, the method including: a step of culturing the Chlamydomonas reinhardtii while applying intermittent light to the Chlamydomonas reinhardtii.
(4) The method for producing an extract of Chlamydomonas reinhardtii as descried in (3), wherein an application time of the intermittent light per 24 hours is 8 hours or longer and 10 hours or shorter.

According to the present invention, it is possible to provide an organic cosmetic containing at least one dipeptide derived from pig placenta.

### DETAILED DESCRIPTION OF THE INVENTION

Hereinafter, an embodiment of the present invention will be described.

### [Cosmetic]

The cosmetic of the present embodiment contains an extract of Chlamydomonas reinhardtii. Here, the extract of the Chlamydomonas reinhardtii contains one or more dipeptides selected from the group consisting of glycine-leucine (Gly-Leu), leucine-glycine (Leu-Gly), glycine-isoleucine (Gly-Ile), and isoleucine-glycine (Ile-Gly). Therefore, an organic cosmetic containing at least one dipeptide derived from pig placenta can be obtained. As a result, it is expected that the use of the cosmetic of the present embodiment will provide the same effects as cosmetics containing a pig placenta extract.

The Chlamydomonas reinhardtii is not particularly limited, and examples thereof include a UTEX 90 strain and a Honda DREAMO strain (accession number: FERM BP-22306). Here, the UTEX 90 strain is the parent strain of the Honda DREAMO strain.

Here, an extract of the Honda DREAMO strain includes Gly-Leu, Leu-Gly, and Ile-Gly.

The form of the cosmetic of the present embodiment is not particularly limited, and examples thereof include a serum. The serum contains, for example, a base material including butylene glycol and water, and an extract of the Chlamydomonas reinhardtii, and the content of the extract of the Chlamydomonas reinhardtii is 1 ppm or more and 10000 ppm or less. Here, the ratio between butylene glycol and water is not particularly limited.

[Method for Producing Extract of Green Alga Chlamydomonas reinhardtii]

The method for producing an extract of the Chlamydomonas reinhardtii of the present embodiment includes a step of culturing the green alga Chlamydomonas reinhardtii while applying intermittent light to the Chlamydomonas reinhardtii. Therefore, the content of the one or more dipeptides selected from the group consisting of glycine-leucine, leucine-glycine, glycine-isoleucine, and isoleucine-glycine in the extract of the Chlamydomonas reinhardtii is increased.

The light period per 24 hours is preferably 8 hours or longer and 10 hours or shorter, and more preferably 8.5 hours or longer and 9.5 hours or shorter. When the light period per 24 hours is 8 hours or longer and 10 hours or shorter, the content of the one or more dipeptides selected from the group consisting of glycine-leucine, leucine-glycine, glycine-isoleucine, and isoleucine-glycine in the extract of the Chlamydomonas reinhardtii is increased.

The wavelength of light to be applied in the light period is not particularly limited, but is, for example, 300 nm or more and 800 nm or less. The photosynthetic photon flux density of the light to be applied in the light period is not particularly limited, but is, for example, 60 μmol/m²/s or more and 400 μmol/m²/s or less. The light source of the light to be applied in the light period is not particularly limited, and examples thereof include a white LED.

The method for culturing the Chlamydomonas reinhardtii is not particularly limited, and examples thereof include static culture, shake culture, submerged culture, and aeration culture.

The medium used for culturing the Chlamydomonas reinhardtii is not particularly limited, and examples thereof include a TAP medium, an N1 medium, a urea medium, an HSM medium, and a BG11 medium.

The temperature when culturing the Chlamydomonas reinhardtii is not particularly limited, but is, for example, 25° C. or higher and 28° C. or lower.

The gas flow rate per unit volume when culturing the Chlamydomonas reinhardtii using aeration culture is not particularly limited, but is, for example, 0.001 VVM or more and 10 VVM or less. VVM indicates a volume ratio of gas that is aerated with respect to the medium in one minute.

When culturing the Chlamydomonas reinhardtii using aeration culture, it is preferable to supply carbon dioxide to the medium. The concentration of carbon dioxide in the gas to be aerated is not particularly limited, but is, for example, 0.1% by volume or more and 10% by volume or less. The supply source of carbon dioxide is not particularly limited, and examples thereof include a carbon dioxide cylinder, a gas discharged from a factory or the like, and concentrated carbon dioxide collected from the atmosphere by direct air capture (DAC).

The method for collecting the cultured Chlamydomonas reinhardtii is not particularly limited, and examples thereof include centrifugation and filtration.

The collected Chlamydomonas reinhardtii may be dried as necessary.

The method for drying the Chlamydomonas reinhardtii is not particularly limited, and examples thereof include freeze-drying, spray-drying, hot air drying, and sun-drying.

The extraction method used for extracting the Chlamydomonas reinhardtii is not particularly limited, and examples thereof include ultrasonic extraction.

The solvent used for extraction of the Chlamydomonas reinhardtii is not particularly limited, and examples thereof include lower alcohols such as methanol, ethanol, propyl alcohol, and isopropyl alcohol; lower aliphatic ketones such as acetone and methyl ethyl ketone; polyhydric alcohols such as 1,3-butylene glycol, propylene glycol, and glycerin; and hydrophilic organic solvents such as dimethyl sulfoxide (DMSO). Among these, DMSO is preferable.

The method for collecting a liquid extract of the Chlamydomonas reinhardtii is not particularly limited, and examples thereof include centrifugation and filtration.

The collected extract of the Chlamydomonas reinhardtii may be dried as necessary.

The method for drying the extract of the Chlamydomonas reinhardtii is not particularly limited, and examples thereof include freeze-drying.

### EXAMPLES

Hereinafter, examples of the present invention will be described, but the present invention is not limited to these examples.

### Example 1

[Culture of Green Alga Chlamydomonas reinhardtii]

A part of a Honda DREAMO strain as Chlamydomonas reinhardtii was collected from a UREA medium, and subjected to aeration culture (seed culture) for 3 to 4 days using 6 L of a UREA medium under the following culture conditions.
Temperature: 25° C. to 28° C.
Light/dark period: light period 8 to 10 hours, dark period 16 to 14 hours
Photosynthetic photon flux density of light to be applied during light period: 60 μmol/m²/s to 400 μmol/m²/s
Light source of light to be applied in light period: white LED
Gas flow rate per unit volume: 0.15 VVM
Concentration of carbon dioxide in gas: 5% by volume

Next, 500 mL of the seed culture was centrifuged at 3000 rpm for 5 minutes, the culture supernatant was removed, and the seed-cultured Honda DREAMO strain was collected.

Next, the collected Honda DREAMO strain was subjected to aeration culture (pre-culture) for 7 days using 6 L of a UREA medium under the same culture conditions as in the seed culture.

Next, the pre-cultured Honda DREAMO strain was subjected to aeration culture (main culture) for 7 days using 1 L of an N1 medium under the same culture conditions as in the seed culture. The aeration conditions were as follows.
Gas flow rate per unit volume: 0.15 VVM
Concentration of carbon dioxide in gas: 5% by volume

Next, 5 L of the main culture was centrifuged at 6000 rpm for 10 minutes, the culture supernatant was removed, and the main-cultured Honda DREAMO strain was collected.

### [Extraction]

About 20 mL of the recovered Honda DREAMO strain was ultrasonically extracted with 100 mL of DMSO for 5 minutes using an ultrasonic homogenizer. At this time, the ultrasonic extraction was carried out on ice.

Next, the mixture of the liquid extract and residue was centrifuged at 3000 rpm for 5 minutes, after which the residue was removed, and about 100 mL of the liquid extract was collected.

Next, the liquid extract was freeze-dried to obtain an extract of the Honda DREAMO strain.

### Comparative Example 1

An extract of the Honda DREAMO strain was obtained in the same manner as in Example 1 except that continuous light was applied instead of intermittent light, that is, the light period was 24 hours.

### [Dipeptide Content in Extract of Honda DREAMO Strain]

The content [g] of dipeptides (Gly-Leu, Leu-Gly, and Ile-Gly) per 1 g (dry weight) of the extract of the Honda DREAMO strain was measured using a high-performance liquid chromatography (HPLC).

Table 1 shows the evaluation results of the dipeptide content in the extract of the Honda DREAMO strain.

TABLE 1

| | | Comparative |
|---|---|---|
| | Example 1 | Example 1 |
| Gly-Leu | 8.70 × 10⁻⁵ | 4.20 × 10⁻⁵ |
| Leu-Gly | 7.00 × 10⁻⁶ | 7.00 × 10⁻⁷ |
| Ile-Gly | 2.90 × 10⁻⁵ | 2.40 × 10⁻⁶ |
| Total | 1.23 × 10⁻⁴ | 4.51 × 10⁻⁵ |

It can be seen from Table 1 that the extract of the Honda DREAMO strain of Example 1 has a higher dipeptide content than the extract of the Honda DREAMO strain of Comparative Example 1.

What is claimed is:

1. A cosmetic comprising: an extract of Chlamydomonas reinhardtii,
the extract of the Chlamydomonas reinhardtii comprising one or more dipeptides selected from the group consisting of glycine-leucine, leucine-glycine, glycine-isoleucine, and isoleucine-glycine.

2. The cosmetic according to claim 1, wherein the Chlamydomonas reinhardtii is a Honda DREAMO strain (accession number: FERM BP-22306).

3. A method for producing an extract of Chlamydomonas reinhardtii,
the extract of the Chlamydomonas reinhardtii comprising one or more dipeptides selected from the group consisting of glycine-leucine, leucine-glycine, glycine-isoleucine, and isoleucine-glycine,
the method comprising: a step of culturing the Chlamydomonas reinhardtii while applying intermittent light to the Chlamydomonas reinhardtii.

4. The method according to claim 3, wherein an application time of the intermittent light per 24 hours is 8 hours or longer and 10 hours or shorter.
